(19) United States
(12) Patent Application Publication
(10) Pub. No.: US 2015/0230745 A1
(43) Pub. Date: Aug. 20, 2015

(54) DEVICE OF DETECTING BLADDER PRESSURE AND METHOD OF JUDGING REMOVAL TIMING OF CATHETER FROM PATIENT'S BODY

(71) Applicant: CHIMEI MEDICAL CENTER, Tainan City (TW)

(72) Inventors: JHI-JOUNG WANG, TAINAN CITY (TW); KUAN-HUA HUANG, TAINAN CITY (TW); BOR-SHYH LIN, TAINAN CITY (TW); JIUN-HUNG LIN, TAINAN CITY (TW)

(73) Assignee: CHIMEI MEDICAL CENTER, Tainan City (TW)

(21) Appl. No.: 14/183,636

(22) Filed: Feb. 19, 2014

(51) Int. Cl. A61B 5/20; A61M 25/00

(52) U.S. Cl. CPC A61B 5/205; A61M 25/0043; A61M 2025/0002; A61F 5/451

(57) ABSTRACT

A device of detecting bladder pressure contains: a tube and a pressure gauge. The tube includes an expansion end and a pressure detection end communicating with the expansion end, the expansion end is a closed end and is adapted to be inserted into a bladder to abut against an inner wall of the bladder.

The pressure gauge is coupled to the pressure detection end of the tube so as to measure the pressure in the bladder when the expansion end is inserted into the bladder. Also, a method of judging removal timing of catheter from patient's body is executed by using the device. Thereby, the doctor is capable of judging whether the catheter can be removed from patient's body.

### FIELD OF THE INVENTION

The present invention relates to a device of detecting bladder pressure and a method of judging removal timing of catheter from patient's body, and more particularly to a device and a method in which a tube is provided, and a first end of the tube is inserted into the bladder so as to sustain pressure in the bladder, and a second end of the tube is used to senses the pressure which will be judged

### BACKGROUND OF THE INVENTION

Conventional catheter cooperates with urodynamic inspection equipment so as to check urodynamic, pressure in bladder, and urinating ability of a patient, such that a doctor can judge whether the catheter can be removed from the patient's body. In use, the catheter is inserted into the patient's body and the urodynamic inspection equipment is moved to close to the patient by medical personnel so as to check the patient's bladder. However, the inspection equipment is in a big size, so it is moved inconveniently. Likewise, the inspection equipment is expensive so it is not popular to be used to inspect the patient's bladder.

China Patent No. 102579029 discloses a catheter having a pressure sensor combined therewith for sensing pressure in bladder, wherein the pressure sensor is arranged on a flow patch of urine. Since the pressure sensor is a mechanical component, it infects the patient easily if not being sterilized completely. Furthermore, the pressure sensor is infected by urine due to it is arranged on the flow patch of urine. To avoid the pressure sensor infecting the patient, the pressure sensor cannot be used repeatedly.

The present invention has arisen to mitigate and/or obviate the afore-described disadvantages.

### SUMMARY OF THE INVENTION

The primary object of the present invention is to provide a device of detecting bladder pressure and a method of judging removal timing of catheter from patient's body by which the urodynamic testing can be performed easily and quickly; especially, a urinating state or pressure in bladder can be checked quickly even when a catheter is inserted into the patient's body. Furthermore, a pressure sensor of the present invention will not contact with the bladder so as to prevent the pressure sensor from pollution.

To obtain the above objective, the present invention provides a device of detecting bladder pressure, which has a tube and a pressure gauge. The tube includes an expansion end and a pressure detection end. The expansion end communicates with the pressure detection end and is a closed end, wherein the expansion end is adapted to be inserted into a bladder to abut against an inner wall of the bladder. The pressure gauge is coupled to the pressure detection end of the tube so as to measure a pressure in the bladder when the expansion end abuts against the bladder.

Preferably, the pressure gauge is coupled to the pressure detection end of the tube via a tee pipe, and wherein the tee pipe has a first end connected to the pressure detection end of the tube, a second end connected to the pressure gauge, and a third end joining with a check valve.

Preferably, the pressure gauge is an electronic pressure gauge and has a recorder for recording a pressure change on the expansion end of the tube.

The present invention also provides a device of detecting bladder pressure, which has a first tube, a second tube, a third tube, and a pressure gauge. The first tube includes a catheterizing end and a discharge end opposite to the catheterizing end. The catheterizing end communicates with the discharge end. The second tube is fitted on an outer peripheral side of the first tube and includes an expansion end and a pressure detection end. The pressure detection end communicates with the expansion end, wherein the expansion end of the second tube is a closed end and is located near the catheterizing end of the first tube, and the expansion end of the second tube is adapted to be inserted into a bladder together with the catheterizing end of the first tube to abut against an inner wall of the bladder. The pressure gauge is coupled to the pressure detection end of the second tube so as to measure a pressure in the bladder when the expansion end abuts against the bladder.

Preferably, the pressure gauge is coupled to the pressure detection end of the second tube via a tee pipe, and wherein the tee pipe has a first end connected to the pressure detection end of the second tube, a second end connected to the pressure gauge, and a third end joining with a check valve.

Preferably, a valve is disposed on the discharge end of the first tube.

Preferably, the pressure gauge is an electronic pressure gauge and has a recorder for recording a pressure change on the expansion end of the second tube.

Preferably, a third tube is connected with the first tube, and a channel is formed between the first tube and the third tube, and wherein the third tube communicates with the first tube via an opening and couples with a check valve.

The present invention further provides a method of judging removal timing of catheter from patient's body by using the device. The method contains steps of: inserting the catheterizing end of the first tube and the expansion end of the second tube into the bladder, feeding fluid or gas into the second tube from the check valve joined with the tee pipe so that the expansion end of the second tube expends to abut against the inner wall of the bladder, thereafter connecting the discharge end of the first tube with a urine collection bag so that the first tube is used as a catheter; and requesting a patient to exert force to simulate urination so that detrusor muscle of the patient is contracted, such that the bladder is compressed by the detrusor muscle to apply a pressure to the expansion end of the second tube, and wherein the pressure gauge senses the pressure on the expansion end and obtains a pressure value, hence a doctor is enabled to judge contractility of the detrusor muscle according to the pressure value to decide removal timing of the catheter from the patient's body. Thereby, the method and the device provided by the present invention have following advantages:

1. The doctor can check urodynamic to the patient easily to avoid moving the patient or transporting the device troublesomely.

2. In addition to be used in combination with a catheter, the second tube of the device can be solely applied to perform urodynamic testing.

3. The pressure gauge senses pressure on the expansion end of the second tube and therefore prevents urine from contacting with the pressure gauge so that the pressure gauge can be used repeatedly and has using hygiene.

### BRIEF DESCRIPTION OF THE DRAWINGS

FIG. 1 is a plan view showing the assembly of a device of detecting bladder pressure according to a first embodiment of the present invention.

FIG. 2 is a cross sectional view of FIG. 1.

FIG. 3 is a cross sectional view showing the operation of the device of detecting bladder pressure according to the first embodiment of the present invention.

FIG. 4 is a cross sectional view showing the assembly and the operation of a device of detecting bladder pressure according to a second embodiment of the present invention.

### DETAILED DESCRIPTION OF THE PREFERRED EMBODIMENTS

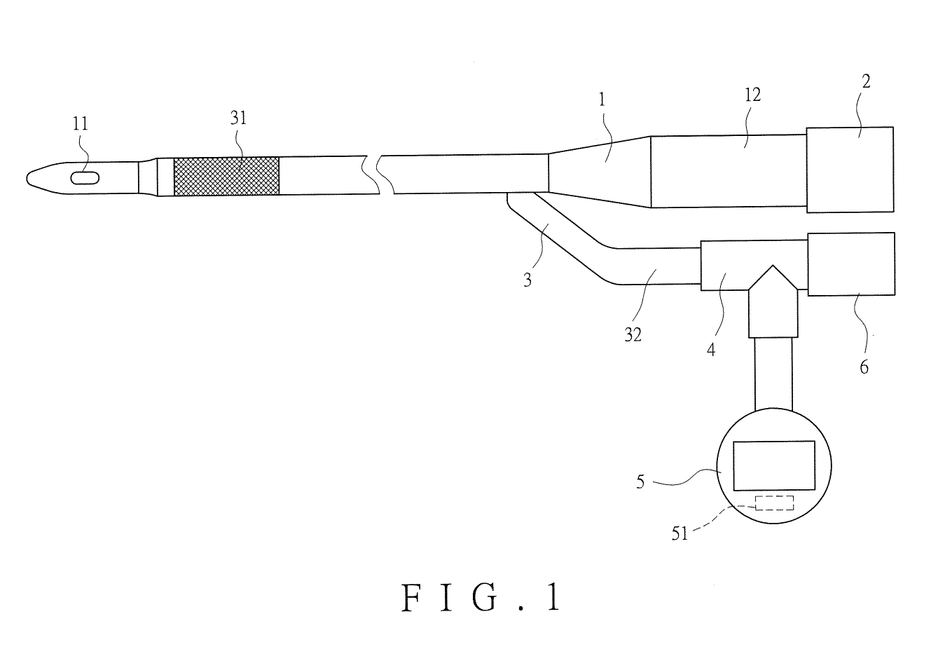

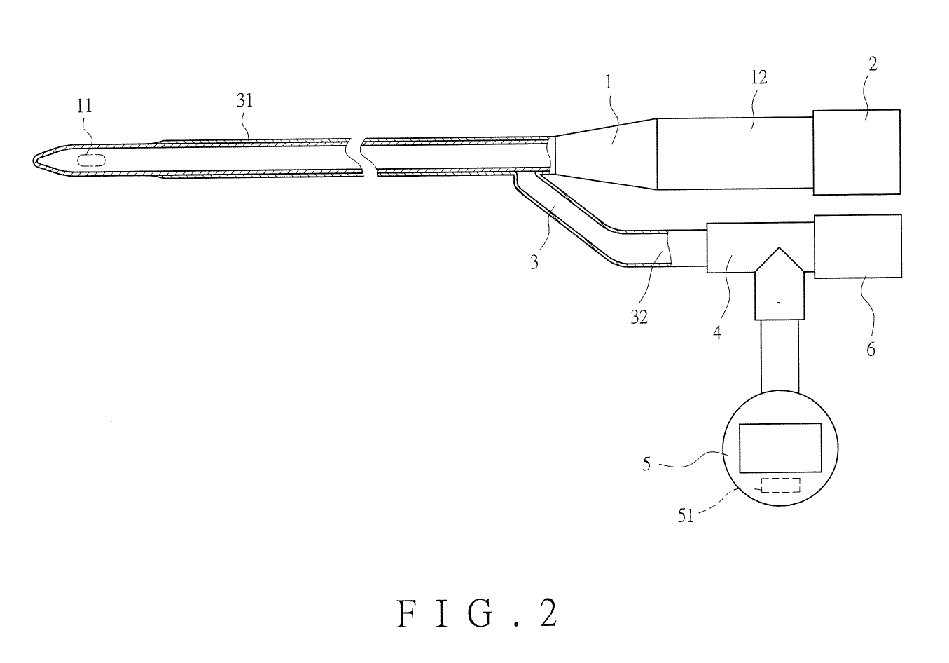

With reference to FIGS. 1 and 2, a device of detecting bladder pressure according to a first embodiment of the present invention has catheter function and comprises a first tube 1, a second tube 3 and a pressure gauge 5.

The first tube 1 includes a catheterizing end 11 and a discharge end 12 which is opposite to the catheterizing end 11. The discharge end 12 communicates with the catheterizing end 11 and has a valve 2 disposed thereon. The second tube 3 is fitted on an outer peripheral side of the first tube 1 and includes an expansion end 31 and a pressure detection end 32. The pressure detection end 32 communicates with the expansion end 31. The expansion end 31 is a closed end and is located near the catheterizing end 11 of the first tube 1. The pressure gauge 5 is coupled to the pressure detection end 32 of the second tube 3 via a tee pipe 4, wherein a first end of the tee pipe 4 is connected to the pressure detection end 32 of the second tube 3, a second end of the tee pipe 4 is connected to the pressure gauge 5, and a third end of the tee pipe 4 is joined to a check valve 6. In this embodiment, the pressure gauge 5 is an electronic pressure gauge and has a recorder 51.

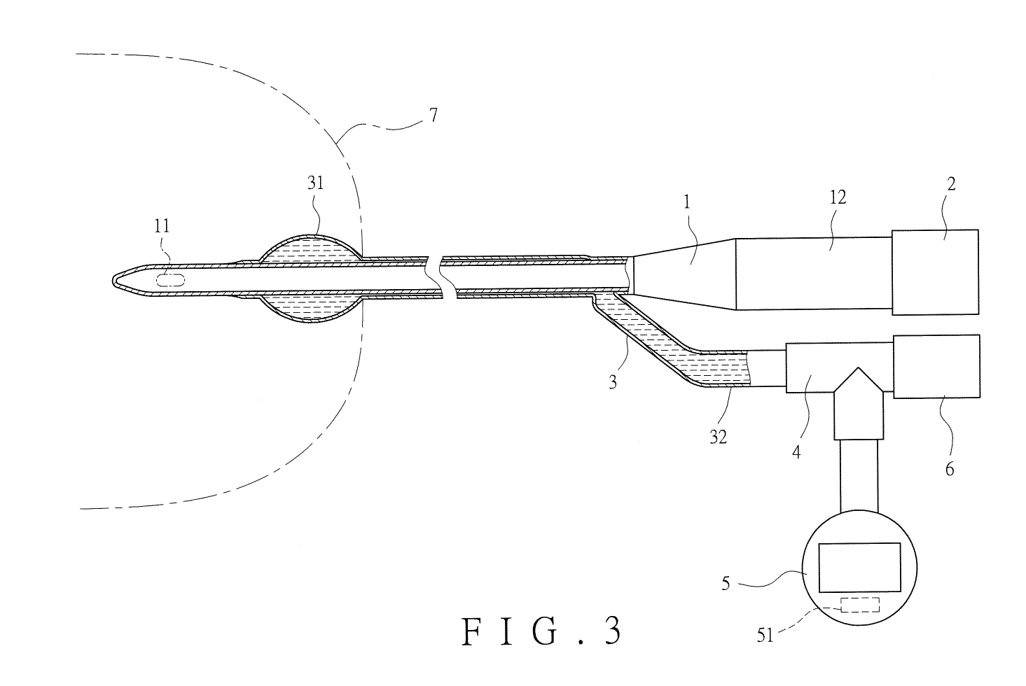

Referring to FIG. 3, the device of detecting bladder pressure can be used to judge whether a patient needs a catheter, and the first tube 1 can be used as the catheter. Specifically, when in use, the catheterizing end 11 of the first tube 1 and the expansion end 31 of the second tube 3 are inserted into bladder 7 of the patient, and fluid (such as saline solution) or gas (such as air) is fed into the second tube 3 from the check valve 6 which is joined to the tee pipe 4 so that the expansion end 31 of the second tube 3 expends to abut against an inner wall of the bladder 7 and thus to be positioned. Thereafter, the discharge end 12 of the first tube 1 is in connection with a urine collection bag (not shown) to be served as the catheter.

Furthermore, when a doctor desires to judge urine production ability of the patient, the valve 2 disposed on the first tube 1 is turned off so that urine is accumulated in the bladder 7 of the patient and thus to apply pressure to the expansion end 31 of the second tube 3, wherein the pressure is changed according to accumulated volume of urine. The pressure gauge 5 senses any change of the pressure in time and stores all pressure values in the recorder 51 thereof, thereafter an average urine production (urine production volume per unit time) is derived from the pressure values by a computer (since such a derivation is a well-known art, further remarks are omitted), such that, with reference to water intake and average urine production of the patient, the doctor is capable of estimating whether the urine production ability of the patient complies with normal standard.

The device of detecting bladder pressure further can be used to judge contractility of detrusor muscle of the patient so as to execute a method of judging removal timing of catheter from patient's body. The method comprises steps of: requesting the patient to exert force to stimulate urination when the expansion end 31 of the second tube 3 is inserted into the bladder 7 such that the detrusor muscle of the patient is contracted, so that the bladder 7 of the patient is compressed to apply pressure to the expansion end 31 of the second tube 3; thereafter, sensing the pressure by the pressure gauge 5 so that a pressure value is stored in the recorder 51 of the pressure gauge 5, hence the doctor is capable of judging if the contractility of the detrusor muscle of the patient complies with normal standard. If so, it is appropriate to remove the catheter from the patient's body.

Thereby, the doctor judges whether the catheter can be removed from the patient's body by using the device of detecting bladder pressure mentioned above instead of conventional urodynamic testing equipment, thus avoiding transportation inconvenience. Furthermore, in addition to be used in combination with a catheter (such as the first tube 1) as mentioned above, the second tube 3 can be solely applied to detect urodynamic.

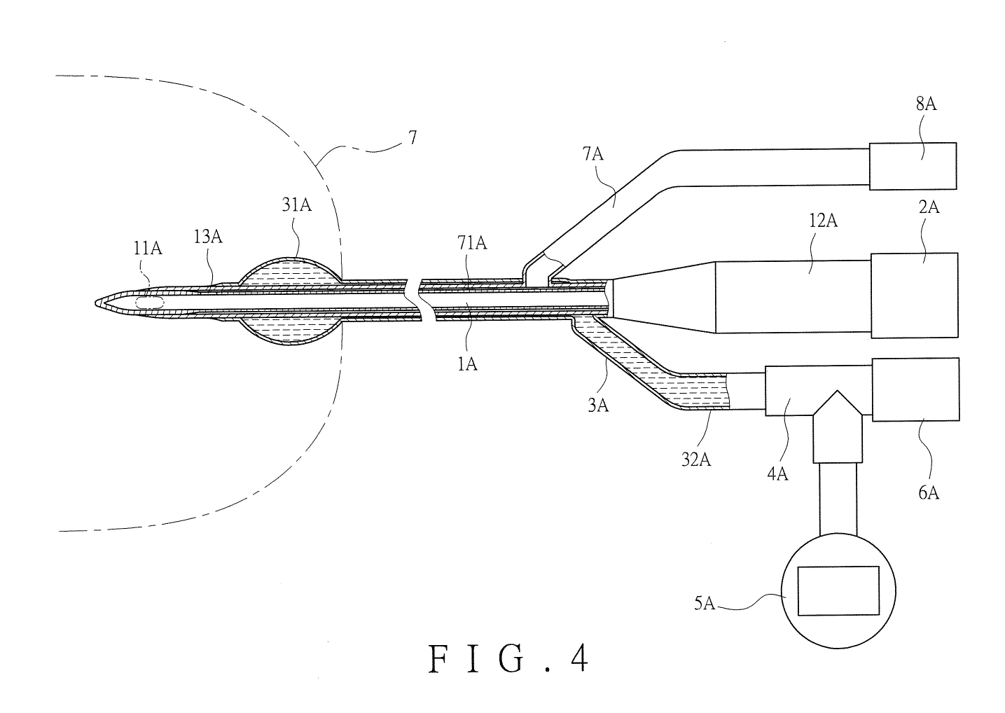

As shown in FIG. 4, a device of detecting bladder pressure according to a second embodiment of the present invention has catheter function and comprises a first tube 1A, a second tube 3A, a third tube 7A, and a pressure gauge 5A.

The first tube 1A includes a catheterizing end 11A and a discharge end 12A opposite to the catheterizing end 11A. The discharge end 12A communicates with the catheterizing end 11A and has a valve 2A disposed thereon. The second tube 3A is fitted on an outer peripheral side of the first tube 1A and includes an expansion end 31A and a pressure detection end 32A which communicates with the expansion end 31A, wherein the expansion end 31A is a closed end and located near the catheterizing end 11A of the first tube 1A. The pressure gauge 5A is coupled to the pressure detection end 32A of the second tube 3A via a tee pipe 4A, wherein a first end of the tee pipe 4A is connected to the pressure detection end 32A of the second tube 3A, a second end of the tee pipe 4A is connected to the pressure gauge 5A, and a third end of the tee pipe 4A is joined with a check valve 6A. The third tube 7A is connected with the first tube 1A and is disposed between the first tube 1A and the second tube 3A, and a channel 71A is formed between the first tube 1A and the third tube 7A. The third tube 7A is also joined to a check valve 8A, and wherein the third tube 7A communicates with the first tube 1A via an opening 13A. In this embodiment, the opening 13A is defined on the first tube 1A.

When in use, the valve 2A disposed on the discharge end 12A of the first tube 1A is turned off after the device of detecting bladder pressure of this embodiment is inserted into the bladder 7 of the patient, and then a saline solution is fed from the check valve 8A joined to the third tube 7A and flows through the channel 71A and the opening 13A and into the catheterizing end 11A of the first tube 1A to fill the bladder 7. Thereafter, the patient is requested to exert force to stimulate urination so that the bladder 7 of the patient is compressed to apply pressure on the expansion end 31A of the second tube 3A. The pressure gauge 5A coupled to the pressure detection end 32A of the second tube 3A senses the pressure and therefore obtains a pressure value from which the urinating ability of the patient when the bladder 7 is filled with urine can be learned.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A device for detecting bladder pressure comprising:
a tube including an expansion end and a pressure detection end, the expansion end communicating with the pressure detection end and being a closed end, wherein the expansion end is adapted to be inserted into a bladder to abut against an inner wall of the bladder; and
a pressure gauge coupled to the pressure detection end of the tube so as to measure a pressure in the bladder when the expansion end is inserted into the bladder.

2. The device as claimed in claim 1, wherein the pressure gauge is coupled to the pressure detection end of the tube via a tee pipe, and wherein the tee pipe has a first end connected to the pressure detection end of the tube, a second end connected to the pressure gauge, and a third end joining with a check valve.

3. The device as claimed in claim 1, wherein the pressure gauge is an electronic pressure gauge and has a recorder for recording a pressure change on the expansion end of the tube.

4. A device for detecting bladder pressure comprising:
a first tube including a catheterizing end and a discharge end opposite to the catheterizing end, the catheterizing end communicating with the discharge end;
a second tube fitted on an outer peripheral side of the first tube and including an expansion end and a pressure detection end, the pressure detection end communicating with the expansion end, wherein the expansion end of the second tube is a closed end and is located near the catheterizing end of the first tube, and the expansion end of the second tube is adapted to be inserted into a bladder together with the catheterizing end of the first tube to abut against an inner wall of the bladder; and
a pressure gauge coupled to the pressure detection end of the second tube so as to measure a pressure in the bladder when the expansion end is inserted into the bladder.

5. The device as claimed in claim 4, wherein the pressure gauge is coupled to the pressure detection end of the second tube via a tee pipe, and wherein the tee pipe has a first end connected to the pressure detection end of the second tube, a second end connected to the pressure gauge, and a third end joining with a check valve.

6. The device as claimed in claim 4 further comprising a valve disposed on the discharge end of the first tube.

7. The device as claimed in claim 4, wherein the pressure gauge is an electronic pressure gauge and has a recorder for recording a pressure change on the expansion end of the second tube.

8. The device as claimed in claim 4 further comprising a third tube, wherein the third tube is connected with the first tube, and a channel is formed between the first tube and the third tube, and wherein the third tube communicates with the first tube via an opening and couples with a check valve.

9. A method of judging removal timing of catheter from patient's body by using the device as claimed in claim 5, comprising steps of:
inserting the catheterizing end of the first tube and the expansion end of the second tube into the bladder, feeding fluid or gas into the second tube from the check valve joined with the tee pipe so that the expansion end of the second tube expends to abut against the inner wall of the bladder, thereafter connecting the discharge end of the first tube with a urine collection bag so that the first tube is used as a catheter; and
requesting a patient to exert force to simulate urination so that detrusor muscle of the patient is contracted, such that the bladder is compressed by the detrusor muscle to apply a pressure to the expansion end of the second tube, and wherein the pressure gauge senses the pressure on the expansion end and obtains a pressure value, hence a doctor is enabled to judge contractility of the detrusor muscl according to the pressure value to decide removal timing of the catheter from a patient's body.
